(19) United States
(12) Patent Application Publication
(10) Pub. No.: US 2025/0212847 A1
(43) Pub. Date: Jul. 3, 2025

(54) A SYSTEM AND METHOD FOR DETERMINING THE READINESS OF SUCKLING MAMMALS FOR WEANING

(71) Applicant: S.C.R. (ENGINEERS) LIMITED, Netanya (IL)

(72) Inventors: Rotem RABINOVITZ, Beit Hashita (IL); Eran GENZEL, Tel-Aviv (IL); Elimelech Mordechai SCHREIBER, Petah Tikva (IL); Eran FRIEDMAN, Kibbutz Ketura (IL)

(73) Assignee: S.C.R. (ENGINEERS) LIMITED, Netanya (IL)

(21) Appl. No.: 18/852,217

(22) PCT Filed: Apr. 4, 2023
(86) PCT No.: PCT/IL2023/050367

§ 371 Date: Sep. 27, 2024

(30) Foreign Application Priority Data
Apr. 5, 2022 (IL) 291969

(51) Int. Cl. A01K 29/00; G16H 50/30

(52) U.S. Cl. CPC A01K 29/005; G16H 50/30

(57) ABSTRACT

The presently disclosed subject matter aims to a system and method directed to determine the readiness of suckling mammals for weaning. The system and method include a processing circuitry configured to: obtain information enabling determination of one or more behaviors of said suckling mammal over time; analyze said information to characterize one or more behaviors exhibited by said suckling mammal over said time; determine whether said one or more characterized behaviors meet a predefined set of rules indicative of a suckling mammal's readiness for weaning; and, upon said one or more characterized behaviors meeting said predefined set of rules, perform weaning of said suckling mammal.

### CROSS-REFERENCE TO RELATED APPLICATIONS

PCT application PCT/IL2021/050797, entitled “A System and Method for Efficient Animal Monitoring Device Power Consumption Management”, filed on Jun. 29, 2021, is incorporated herein by reference in its entirety.

### TECHNICAL FIELD

The present invention relates to the field of animal health monitoring, including systems and methods for determining the readiness of suckling mammals for weaning.

### BACKGROUND

Mammals are a group of warm-blooded vertebrate animals constituting the class Mammalia, characterized by the presence of mammary glands, which in females produce milk for nursing (nourishing) their offspring. The nursing period of mammals' offspring varies between family members, ranging from just four days in hooded seals up to about 6-8 years in orangutans.

During the nursing period, the offspring draws milk from the nipple or teat of his mother's mammary gland/s into his mouth in an act called suckling. The transition process of gradually moving the suckling offspring from a diet that is based on milk produced by its mother to what will be its mature diet is called weaning.

Though all mammals eventually wean off of nursing, the particular point in time in which each given suckling mammal is ready to be weaned remains obscure. Currently, in cases involving large groups of mammals such as cattle, weaning of suckling calves is performed according to characteristics, such as age, height and weight, regardless of each individual calf's readiness. This may potentially cause calves that are weaned prematurely to exhibit abnormal and/or unhealthy behaviors that may influence their well-being, normal growth, and/or productivity.

Thus, there is a need in the art for a new system and method for determining the readiness of suckling mammals for weaning.

### GENERAL DESCRIPTION

In accordance with a first aspect of the presently disclosed subject matter, there is provided a system for determining the readiness of a suckling mammal for weaning, the system comprising a processing circuitry configured to: obtain information enabling determination of one or more behaviors of the suckling mammal over time; analyze the information to characterize one or more behaviors exhibited by the suckling mammal over the time; determine whether the one or more characterized behaviors meet a predefined set of rules indicative of a suckling mammal's readiness for weaning; and, upon the one or more characterized behaviors meeting the predefined set of rules, perform weaning of the suckling mammal.

In one embodiment of the presently disclosed subject matter and/or embodiments thereof, each rule of the predefined set of rules is associated with at least one behavior of the one or more behaviors.

In one embodiment of the presently disclosed subject matter and/or embodiments thereof, each rule is determined by analyzing historical information related to the at least one behavior associated with the rule.

In one embodiment of the presently disclosed subject matter and/or embodiments thereof, the historical information related to the at least one behavior is obtained from one or more mammal populations.

In one embodiment of the presently disclosed subject matter and/or embodiments thereof, the one or more mammal populations include a herd of the suckling mammal.

In one embodiment of the presently disclosed subject matter and/or embodiments thereof, the one or more behaviors of the suckling mammal includes any of: rumination, suckling, grazing, eating, walking, resting, non-nutritive suckling, and heavy breathing.

In accordance with a second aspect of the presently disclosed subject matter, there is provided a method for determining the readiness of a suckling mammal for weaning comprising: obtaining information enabling determination of one or more behaviors of the suckling mammal over time,; analyzing the information to characterize one or more behaviors exhibited by the suckling mammal over the time; determining whether the one or more characterized behaviors of the suckling mammal meet a predefined set of rules indicative of a suckling mammal's readiness for weaning; and, upon the one or more characterized behaviors meeting the predefined set of rules, performing weaning of the suckling mammal.

In one embodiment of the presently disclosed subject matter and/or embodiments thereof, each rule of the predefined set of rules is associated with at least one behavior of the one or more behaviors.

In one embodiment of the presently disclosed subject matter and/or embodiments thereof, each rule is determined by analyzing historical information related to the at least one behavior associated with the rule.

In one embodiment of the presently disclosed subject matter and/or embodiments thereof, the historical information related to the at least one behavior is obtained from one or more mammal populations.

In one embodiment of the presently disclosed subject matter and/or embodiments thereof, the one or more mammal populations include a herd of the suckling mammal.

In one embodiment of the presently disclosed subject matter and/or embodiments thereof, the one or more behaviors of the suckling mammal includes any of: rumination, suckling, grazing, eating, walking, resting, non-nutritive suckling, and heavy breathing.

In accordance with a third aspect of the presently disclosed subject matter, there is provided a non-transitory computer readable storage medium having computer readable program code embodied therewith, the computer readable program code, executable by at least one processor to perform a method for determining the readiness of a suckling mammal for weaning, the method comprising: obtaining information enabling determination of one or more behaviors of the suckling mammal over time; analyzing said information to characterize one or more behaviors exhibited by said suckling mammal over said time; determining whether the one or more characterized behaviors of the suckling mammal meet a predefined set of rules indicative of a suckling mammal's readiness for weaning; and, upon the one or more characterized behaviors meeting the predefined set of rules, performing weaning of the suckling mammal.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1 is a schematic illustration of an environment in which the system for determining the readiness of suckling mammals for weaning operates, in accordance with the presently disclosed subject matter;

FIG. 2 is a block diagram schematically illustrating one example of a system for determining the readiness of suckling mammals for weaning, in accordance with the presently disclosed subject matter; and,

FIG. 3 is a flowchart illustrating one example of a sequence of operations carried out by a system for determining the readiness of suckling mammals for weaning, in accordance with the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “obtaining”, “determining”, “analyzing”, “performing”, or the like, include action and/or processes of a computer that manipulate and/or transform data into other data, said data represented as physical quantities, e.g., such as electronic quantities, and/or said data representing the physical objects. The terms “computer”, “processor”, “processing resource”, “processing circuitry”, and “controller” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field programmable gate array (FPGA), an application specific integrated circuit (ASIC), etc.), a group of multiple physical machines sharing performance of various tasks, virtual servers co-residing on a single physical machine, any other electronic computing device, and/or any combination thereof.

The operations in accordance with the teachings herein may be performed by a computer specially constructed for the desired purposes or by a general-purpose computer specially configured for the desired purpose by a computer program stored in a non-transitory computer readable storage medium. The term “non-transitory” is used herein to exclude transitory, propagating signals, but to otherwise include any volatile or non-volatile computer memory technology suitable to the application.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

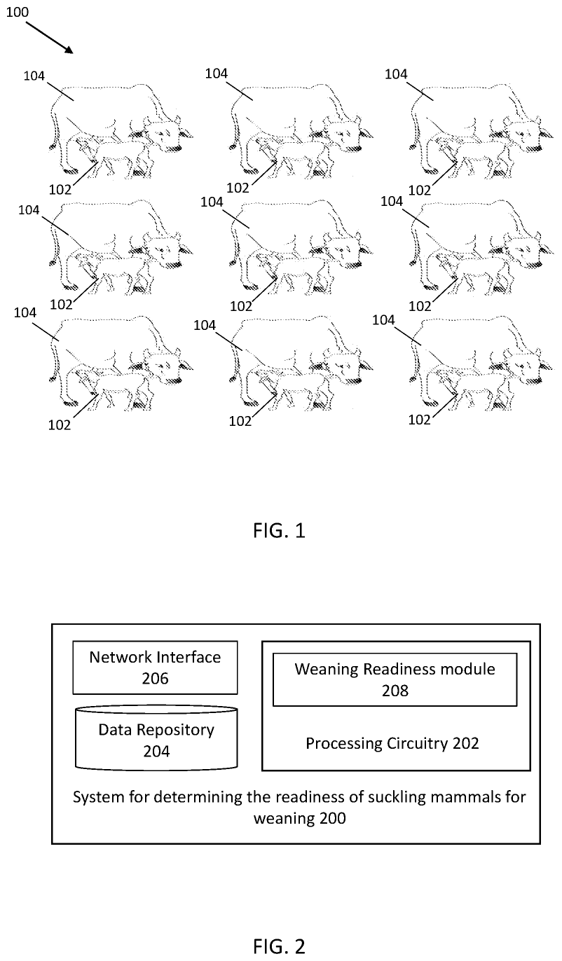

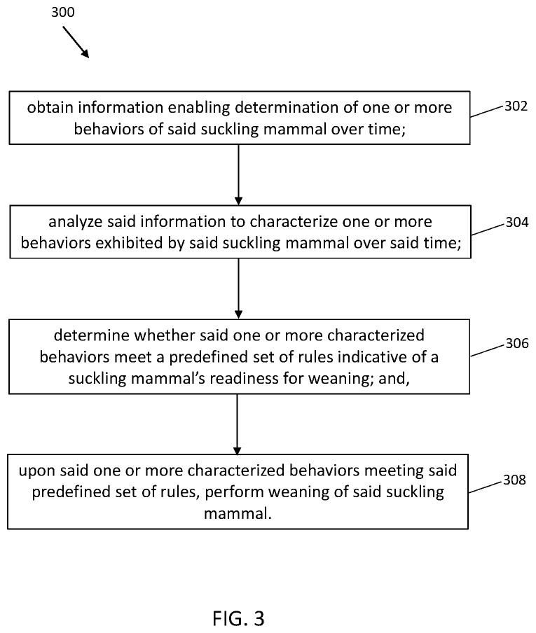

In embodiments of the presently disclosed subject matter, fewer, more and/or different stages than those shown in FIG. 3 may be executed. In embodiments of the presently disclosed subject matter one or more stages illustrated in FIG. 3 may be executed in a different order and/or one or more groups of stages may be executed simultaneously. FIGS. 1 and 2 illustrate a general schematic of the system architecture in accordance with an embodiment of the presently disclosed subject matter. Each module in FIG. 2 can be made up of any combination of software, hardware and/or firmware that performs the functions as defined and explained herein. The modules in FIG. 2 may be centralized in one location or dispersed over more than one location. In other embodiments of the presently disclosed subject matter, the system may comprise fewer, more, and/or different modules than those shown in FIG. 2.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

Bearing this in mind, attention is drawn to FIG. 1, showing a schematic illustration of an operation of a system and method for determining the readiness of suckling mammals for weaning (also interchangeably referred to herein as “system” or “method”), in accordance with the presently disclosed subject matter.

As shown in the schematic illustration, environment 100, which can be a controlled environment, such as a pen or a barn, an uncontrolled environment, such as a meadow or a field, or a combination thereof, includes a mammal population (e.g., a herd) in which one or more mammals are suckling mammals 102 (e.g., calves, foals, kids, and the like). Each of said suckling mammals 102 can be nourished by a respective mature female mammal 104 (the suckling mammal's mother), capable of producing milk through her mammary glands, and/or by other means, such as milk-producing systems, capable of providing milk substitutes and/or milk derived from mature female mammals.

Of the one or more suckling mammals 102 found in environment 100, at least some suckling mammals can be associated with one or more devices (such as tags (e.g., ear tags, neck tags, etc.), collars (e.g., neck collars, etc.), internal implants (e.g., a microchip implant, etc.), and the like) and/or one or more systems (e.g., vision systems, etc.) capable of acquiring information associated with said suckling mammals.

In cases involving using one or more devices, each of said devices can include a sensing mechanism, associated with the device's power source, or its own dedicated power source. The sensing mechanism may contain one or more sensors (e.g., a vibration sensor, a temperature sensor, a velocity sensor, an acceleration sensor (e.g., an accelerometer), a gyroscope, a magnetometer, a pedometer, a location sensor (e.g., a Global Positioning System receiver), a heart rate sensor, a moisture sensor, etc.) configured to collect information relating to a given suckling mammal. The information collected by said sensors can include, for example: (a) behavioral information associated with behaviors exhibited by the suckling mammal, such as rumination, suckling, grazing, eating, walking, resting, heavy breathing, etc., (b) physiological information collected from the suckling mammal's body, such as its temperature, heart rate, biometric information, velocity, acceleration (optionally directional acceleration), spatial orientation, etc., (c) environmental information associated with the environment of the suckling mammal, such as the ambient temperature, ambient moisture, etc., (d) geospatial information associated with the positioning of the suckling mammal, such as the suckling mammal's geographical location, relative location, etc.

It should be noted that the types of information listed above serve as mere examples and that other types of information, alternatively or additionally to the ones listed above, may also be collected by said sensors.

Further to the above, the sensing mechanism may operate according to several operation modes, each defining at least one sensor operation parameter potentially affecting the sensor's power consumption ranges and/or its average/median power consumption. In one example, the sensor operation parameter may be the sensor's sampling rate. In another example, the sensor operation parameter may be the sensor's sensitivity. In yet another example, the sensor operation parameter may be one of: the sensor's dynamic range, the sensor's accuracy, or the sensor's bandwidth.

It should be noted that the types of sensor operation parameters listed above serve as mere examples and that other types of sensor operation parameters, alternatively or additionally to the ones listed above, may also be applicable.

By way of a non-limiting example (presented merely for purposes of better understanding the disclosed subject matter and not in any way intended to limit its scope), a herd of eighteen dairy cows, including nine suckling calves 102 and nine mature female cows 104, is dispersed in a field 100. Each suckling calf of the nine suckling calves 102 is associated with a respective mature female cow of the mature female cows 104 and has an ear tag coupled to its ear. The ear tag of each of the nine suckling calves 102 detects behavioral information associated with rumination, eating, and suckling behaviors exhibited by the respective suckling calf.

Attention is now drawn to a further description of the components of the system for determining the readiness of suckling mammals for weaning 200.

FIG. 2 is a block diagram schematically illustrating one example of the system for determining the readiness of suckling mammals for weaning 200, in accordance with the presently disclosed subject matter.

In accordance with the presently disclosed subject matter, the system for determining the readiness of suckling mammals for weaning 200 (also interchangeably referred to herein as “system 200”) can comprise a network interface 206. The network interface 206 (e.g., a network card, a Wi-Fi client, a Li-Fi client, 3G/4G client, or any other component), enables system 200 to communicate over a network with external systems and handles inbound and outbound communications from such systems. For example, system 200 can receive, through network interface 206, behavioral information associated with one or more behaviors of suckling mammal/s.

System 200 can further comprise or be otherwise associated with a data repository 204 (e.g., a database, a storage system, a memory including Read Only Memory-ROM, Random Access Memory—RAM, or any other type of memory, etc.) configured to store data. Some examples of data that can be stored in the data repository 204 include:
Information enabling determination of one or more behaviors of one or more suckling mammals over time;
One or more characterized behaviors exhibited by one or more suckling mammals over time;
One or more sets of rules indicative of a suckling mammal's readiness for weaning;
Benchmark/s or threshold/s associated with the one or more behaviors exhibited by one or more suckling mammals;
Historical information associated with one or more behaviors exhibited by one or more members of one or more mammal populations; etc.

Data repository 204 can be further configured to enable retrieval and/or update and/or deletion of the stored data. It is to be noted that in some cases, data repository 204 can be distributed, while the system 200 has access to the information stored thereon, e.g., via a wired or wireless network to which system 200 is able to connect (utilizing its network interface 206).

System 200 further comprises processing circuitry 202. Processing circuitry 202 can be one or more processing units (e.g., central processing units), microprocessors, microcontrollers (e.g., microcontroller units (MCUs)) or any other computing devices or modules, including multiple and/or parallel and/or distributed processing units, which are adapted to independently or cooperatively process data for controlling relevant system 200 resources and for enabling operations related to system's 200 resources.

The processing circuitry 202 comprises a weaning readiness module 208, configured to perform a weaning readiness determination process, as further detailed herein, inter alia with reference to FIG. 3.

Turning to FIG. 3 there is shown a flowchart illustrating one example of the operations carried out by the system for determining the readiness of suckling mammals for weaning 200, in accordance with the presently disclosed subject matter.

Accordingly, the system for determining the readiness of suckling mammals for weaning 200 (also interchangeably referred to hereafter as “system 200”) can be configured to perform a weaning readiness determination process 300, e.g., using weaning readiness module 208.

For this purpose, system 200 obtains information enabling determination of one or more behaviors of a given suckling mammal 102 over time (block 302). In one example, said information may include one or more durations of one or more behaviors displayed by the given suckling mammal within a predefined period of time (e.g., 24-hours period). In another example, additionally or alternatively to the example above, said information may include an order in which said one or more behaviors are exhibited, during the predefined period of time.

Next, system 200 analyzes the obtained information to characterize one or more behaviors exhibited by the given suckling mammal 102 over said time (block 304). The one or more characterized behaviors can optionally be, for example, one or more of: rumination, suckling, grazing, eating, walking, resting, heavy breathing, etc.

Once characterized, system 200 determines whether the one or more characterized behaviors meet a predefined set of rules indicative of a suckling mammal's readiness for weaning (block 306).

Each of the rules composing the predefined set of rules may be associated with at least one behavior of the one or more behaviors exhibited by the given suckling mammal. In one example, each rule may involve a benchmark or a threshold related to a given behavior of the one or more behaviors exhibited by the given suckling mammal. Each benchmark or threshold may be determined by analyzing historical information associated with the given behavior in members of one or more mammal populations (e.g., the mammal population (i.e., the herd) of the given suckling mammal, one or more mammal populations of the same type of the given suckling mammal, one or more mammal populations of the same breed of the given suckling mammal, etc.), and may serve as a reference point representing the standard of the behavior in the one or more animal populations. The benchmark or threshold of each given behavior may be, for example, the average or median value of the behavior duration during a specific period of time.

In another example, alternatively, or additionally to the example above, each rule may involve an absolute value representing, for example, a minimal value of a given behavior of the one or more behaviors exhibited by the given suckling mammal (e.g., the minimal duration the given behavior should be exhibited by the given suckling mammal during a specific time period). Each absolute value may be determined by analyzing historical information associated with the given behavior in members of one or more mammal populations (e.g., the mammal population (i.e., the herd) of the given suckling mammal, one or more mammal populations of the same type of the given suckling mammal, one or more mammal populations of the same breed of the given suckling mammal, etc.).

It is to be noted that the examples above are not intended in any way to limit the scope of the presently disclosed subject matter and that the predefined set of rules may consist of only rules involving benchmarks or thresholds, of only rules involving absolute values, or any combination of them.

It is to be further noted that in some cases, the predefined set of rules may also include rules directed to identify correlations between exhibited behaviors of each suckling mammal (e.g., a correlation involving an increase in ruminating behavior with the decrease in suckling behavior, and the like).

Upon the one or more characterized behaviors meeting the predefined set of rules, system 200 performs weaning of the given suckling mammal 102 (block 308).

By way of a non-limiting example and in accordance with the example described hereinbefore in relation to FIG. 1, system 200 obtains information enabling determination of one or more behaviors of each suckling calf of the nine suckling calves 102. System 200 analyzes the obtained information and characterizes three behaviors, ruminating, eating, and suckling, exhibited by each suckling calf during a 24-hours period. System 200 then compares each of the three characterized behaviors to a set of three rules indicative of a suckling mammal's readiness for weaning. The set of three rules includes: (i) a first rule associated with a rumination benchmark representing the average duration members of the herd of eighteen dairy cows spends ruminating, (ii) a second rule associated with an absolute value representing the minimal duration members of the herd of eighteen dairy cows spend eating, and (iii) a third rule associated with a suckling benchmark representing the median duration the herd of eighteen dairy cows spends suckling.

From the comparison, system 200 determines that only four calves of the nine suckling calves 102 are fitted to be weaned, as their durations of rumination, eating, and suckling exceed the rumination benchmark associated with the first rule, the absolute value associated with the second rule, and the suckling benchmark associated with the third rule, respectively.

As a result of determining which suckling mammals are fitted for weaning, system 200 also identifies subgroups (e.g., an individual or a group of individuals) within the plurality of suckling mammals 102, which deviates from the expected growth distribution at a given point in time. The identification of these subgroups can also be a target of system 200, such that, once these subgroups are identified, system 200 can suggest an action directed to enable returning these subgroups to what is considered the expected growth distribution. For example, upon identification of these subgroups, system 200 can suggest entering the identified subgroups to customized management, which may include customized conditions (e.g., having longer feeding time during the day, having more meals provided to them during the day, having less resting time during the day, and the like), for a pre-defined or dynamic time period (e.g., days, weeks, months, etc.) at the end of which they will return to what is considered the expected growth distribution.

By way of example and in accordance with the example described therebefore, the five suckling calves of the nine suckling calves 102, which at the time of decision were found as unfit for weaning by system 200, are entered into a customized program in which each of them will have additional time of rumination, eating, and suckling, so as to return them to be within the expected growth distribution by the end of the customized program duration.

In some cases, the subgroups deviating from the expected growth distribution can include mammals exhibiting high morbidity during a set period of time. These mammals, identified by determining their overall health, e.g., by analyzing historical information associated with behaviors exhibited by each of them during the set period of time, are inherently considered as deviating from the expected growth distribution since they did not have the optimal conditions to maintain the expected growth during said period of time.

It is to be noted, with reference to FIG. 3, that some of the blocks can be integrated into a consolidated block or can be broken down to a few blocks and/or other blocks may be added. It is to be further noted that some of the blocks are optional. It should be also noted that whilst the flow diagram is described also with reference to the system elements that realizes them, this is by no means binding, and the blocks can be performed by elements other than those described herein.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed method. The presently disclosed subject matter further contemplates a machine-readable memory tangibly embodying a program of instructions executable by the machine for executing the disclosed method.

1. A system for identifying at least one suckling mammal within a plurality of suckling mammals, deviating from an expected growth distribution, the system comprising a processing circuitry configured to:
obtain information enabling determination of one or more behaviors of the at least one suckling mammal over time;
analyze the obtained information so as to characterize one or more behaviors exhibited by said at least one suckling mammal over said time;
determine whether the one or more characterized behaviors meet a predefined set of rules, associated with the expected growth distribution; and,
upon said characterized behaviors failing to meet said predefined set of rules, identify said at least one suckling mammal as deviating from said expected growth distribution.

2. The system of claim 1, wherein each rule of the predefined set of rules is associated with at least one behavior of the one or more behaviors.

3. The system of claim 2, wherein each rule is determined by analyzing historical information related to the at least one behavior associated with said rule.

4. The system of claim 3, wherein the historical information is obtained from one or more mammal populations.

5. (canceled)

6. The system of claim 1, wherein the one or more behaviors of the suckling mammal includes any of: rumination, suckling, grazing, eating, walking, resting, non-nutritive suckling, and heavy breathing.

7.-13. (canceled)

14. The system of claim 1, wherein upon identifying said at least one suckling mammal as deviating from said expected growth distribution, said system is configured to suggest at least one action directed to enable returning said suckling mammal to said expected growth distribution.

15. The system of claim 14, wherein said action entails entering said at least one sucking mammal into customized management, following which said sucking mammal is configured to be within said expected growth distribution.

16. The system of claim 1, wherein said predefined set of rules includes rules directed to identify correlations between behaviors exhibited by said at least one suckling mammal.
